(19) United States
(12) Patent Application Publication
(10) Pub. No.: US 2023/0298770 A1
(43) Pub. Date: Sep. 21, 2023

(54) SYSTEMS AND PROCESSES FOR CRITICAL DATA DISPLAY

(71) Applicants: John P. FORD, Unadilla, NY (US); Gustavo P. SUDRE, Washington, DC (US); Liying HUANG, State College, PA (US)

(72) Inventors: John P. FORD, Unadilla, NY (US); Gustavo P. SUDRE, Washington, DC (US); Liying HUANG, State College, PA (US)

(21) Appl. No.: 18/184,969

(22) Filed: Mar. 16, 2023

Related U.S. Application Data
(60) Provisional application No. 63/269,406, filed on Mar. 16, 2022.

(51) Int. Cl. G16H 80/00; G16H 10/40; G16H 10/60

(52) U.S. Cl. CPC G16H 80/00; G16H 10/40; G16H 10/60

(57) ABSTRACT

A critical data display process includes a computer directing a medical testing result for a patient to appear on a health care practitioner critical data display of a health care practitioner display monitor of a single health care practitioner. The process also includes the computer prompting the single health care practitioner through the health care practitioner critical data display to review the medical testing result for the patient on the health care practitioner critical data display, to designate or accept a previous designation of the medical testing result as either critical or noncritical, and to enter or authorize patient instructions. The process further includes the computer directing a patient critical data display to appear on a patient display monitor of a patient representative of the patient. The patient critical data display displays the medical testing result as either critical or noncritical as designated by the single health care practitioner and displays the patient instructions from the single health care practitioner.

### CROSS-REFERENCE TO RELATED APPLICATIONS

This application claims priority to and the benefit of U.S. Provisional Application No. 63/269,406, filed Mar. 16, 2022, entitled “SYSTEMS AND PROCESSES FOR CRITICAL DATA DISPLAY”, which is hereby incorporated herein by reference in its entirety.

### FIELD OF THE INVENTION

The present disclosure is directed to the fields of evaluation, communication, and/or display of information. More particularly, the present disclosure pertains to computer-implemented systems, methods, and displays that facilitate communication between patient representatives and their health care practitioners, especially with regard to medical testing results and, more specifically, critical medical testing results.

### BACKGROUND OF THE INVENTION

Among other problems in the medical field, potentially-avoidable patient medical crises may occur, in part, because critical medical testing results are not recognized or acted upon quickly enough.

Caring for one's family is hard enough. Understanding lab results and communicating with the doctor using conventional systems only adds to the stress. Patients and family members who care for them want a consistent way to view health records, critical lab results brought quickly to their attention, critical ranges that are set by the doctor and tailored to the patient's unique situation and medical history, and doctor instructions delivered with no delay.

Similarly, caring for one's patients is hard. Signing off on lab results and communicating with patients using conventional systems also adds to the stress. Health care providers want a fast, simple, and efficient way to review a large amount of lab results, critical results brought to their attention immediately, critical ranges to be tailored to the unique health conditions of each patient, and a convenient way to communicate instructions to patients or their family or caregivers in real time.

Critical medical testing results, in contrast to abnormal but not critical results, may get lost in an overwhelming volume of noncritical results. Critical results may also be delivered directly to the patient, who may not respond to them well or appropriately. Because these medical testing results may come from laboratories that are near the patient but that may not be linked electronically to the health care practitioner, the health care practitioner may not be aware of the medical testing results until significantly later or even at all unless contacted by the patient.

When confronting a major illness or other medical issue, patients and/or their health care proxies become more easily frustrated and look more to their primary provider for guidance. Conventional medical software systems and electronic health care communication systems are not designed to improve communication between the patient and their primary provider and may exacerbate the patient's frustration. A health care proxy may not even have direct access to the patient's electronic medical record.

Conventional medical software systems and electronic health care communication systems are often complex. Finding where critical information resides in such systems can be difficult. In conventional medical data systems, there is no simple way to establish that one problem or issue has been appropriately taken care of by a health care practitioner, whereas another problem or issue to be addressed by the same or another health care practitioner may still be pending a resolution.

The direct communication of a “critical” result between a patient and the patient's provider may be compromised by a number of factors that obstruct optimal direct communication between the patient and the provider and thus compromise optimal patient care and satisfaction as well as patient safety. These factors may include but are not necessarily limited to the following:
1. Health Insurance Portability and Accountability Act (HIPAA) requirements demand that the patient give prior consent to permit the sharing of the patient's data.
2. Many medical information systems do not interact directly with one another. Thus reports, even critical reports, from a system outside the primary information system link between patient and health care practitioner may remain unknown to the health care practitioner. For example, patient emergency room (ER) visit data may go to a different health care system than that of the patient's health care practitioner.
3. Health care practitioners using existing medical record systems are not enabled to adjust critical ranges to meet the specific needs of the individual patient for his or her specific pre-existing conditions or medical history.

Evaluating medical testing results and communicating critical results to patients within conventional medical software systems is also difficult. A typical patient has no idea as to which medical testing results are critical for that patient based on his or her medical history, and medical testing results sent from the lab directly to the patient leave the patient with no guidance as to the meaning of the testing results or what further actions should be taken. This environment leads to unnecessary added stress for both the health care practitioner and the patient and can distort the judgment and the confidence of both the health care practitioner and the patient. Even health care practitioners struggle as patients trying to navigate electronic health care communication systems (see, for example, Gregg, “The Care I Needed”, New England Journal of Medicine, Vol. 386, pp. 411-413, 2022), leading to avoidable anxiety and stress.

Some medical entities, such as the Mayo Clinic and other health care systems, use dedicated physicians as “concierge” physicians to help patients navigate through the health care system to address the patient's specific needs, but this comes at a considerable expense.

### BRIEF DESCRIPTION OF THE INVENTION

In some embodiments, a computer-implemented process and display improves the communication between patient representative and concierge-doctor-health care practitioner about the patient's critical results from medical tests. The process and display are patient-driven. The patient owns the medical data and, through the process and display, selects which health care practitioners to share it with, thereby satisfying Health Insurance Portability and Accountability Act (HIPAA) regulations. The process and display gather and permit access to records across multiple electronic medical record (EMR) systems. The process and display facilitate the interaction by putting the health care practitioner in a concierge position, such that the health care practitioner can set critical test result values through the process and display that are specific for the patient based on the patient's history. The patient representative benefits through real-time communications with the health care practitioner through the single-screen display interface and knows how to act when critical results come up, such as, for example going to an emergency room for treatment.

In exemplary embodiments, the computer-implemented process and display improves the mutual understanding of both the health care practitioner and the patient representative by providing an improved, consistent interface for correspondence while still accommodating certain individual preferences of both the health care practitioner and the patient representative.

In some embodiments, when a patient has medical testing done, the computer-implemented system allows the patient to decide whether or not to have the results of that testing sent to a single predetermined health care practitioner (e.g., doctor/nurse/specialist) based on whether or not the patient provides predetermined contact information (for example, a fax number or an email address) to the testing lab or facility.

In some embodiments, if the patient provides the predetermined contact information, the computer-implemented system directly receives the patient's electronic medical testing results from the lab conducting the medical testing based on that contact information, thereby satisfying HIPAA regulations. The system receives, digitizes, and categorizes the electronic test results as either within normal ranges or outside of normal ranges (i.e., “critically abnormal”) based on either a standard or customized criterion for criticality and then transmits the test results electronically to the single predetermined health care practitioner designated by the patient in the computer-implemented system and displays the results on a screen to the health care practitioner with any critically abnormal result being highlighted. The health care practitioner reviews the displayed results, optionally adjusts the critical value for any testing results to customize for the patient, approves the displayed results, and provides instructions (based on the test results) for the patient representative to the system. Thus, the computer-implemented system gives the health care practitioner the authority and independence/flexibility to take care of patients' specific needs, a level of autonomy not currently offered in healthcare.

In some embodiments, the computer-implemented system then displays only the single health care practitioner-approved critically abnormal results on a screen of the patient representative along with patient instructions from the health care practitioner (i.e., whether and what further actions the patient should take).

In an exemplary embodiment, a critical data display process includes a computer directing a medical testing result for a patient to appear on a health care practitioner critical data display of a health care practitioner display monitor of a single health care practitioner. The process also includes the computer prompting the single health care practitioner through the health care practitioner critical data display to review the medical testing result for the patient on the health care practitioner critical data display, to designate or accept a previous designation of the medical testing result as either critical or noncritical, and to enter or authorize patient instructions. The process further includes the computer directing a patient critical data display to appear on a patient display monitor of a patient representative of the patient. The patient critical data display displays the medical testing result as either critical or noncritical as designated by the single health care practitioner and displays the patient instructions from the single health care practitioner.

In another exemplary embodiment, a critical data display process includes a computer prompting a patient representative through a patient display to authorize transmittal of medical records to a single health care practitioner. The process also includes the computer electronically receiving a medical testing result for the patient. The process further includes the computer electronically transmitting the medical testing result to the single health care practitioner.

In another exemplary embodiment, a critical data display system includes a server computer. The server computer is configured to electronically correspond with a heath care practitioner computer to receive a medical testing result reviewed and designated as critical or noncritical by a single health care practitioner and to receive a patient instruction provided by the single health care practitioner. The server computer is also configured to direct a patient critical data display to appear on a patient display monitor of a patient representative computer. The server computer is further configured to display the medical testing result on the patient critical data display as critical or noncritical as designated by the single health care practitioner. The server computer is yet further configured to display the patient instruction on the patient critical data display.

In another exemplary embodiment, a critical data display system includes a patient representative computer operatively connected to a patient display monitor. The patient representative computer is configured to electronically correspond with a server computer to receive a medical testing result reviewed and designated as critical or noncritical by a single health care practitioner along with a patient instruction provided by the single health care practitioner. The patient representative computer is also configured to direct a patient critical data display to appear on the patient display monitor of a patient representative of a patient. The patient representative computer is further configured to display the medical testing result on the patient critical data display as critical or noncritical, as designated by the single health care practitioner. The patient representative computer is yet further configured to display the patient instruction on the patient critical data display.

In yet another exemplary embodiment, a critical data display system includes a health care practitioner computer operatively connected to a health care practitioner display monitor. The health care practitioner computer is configured to electronically correspond with a server computer to receive a medical testing result for a patient from a testing facility based on predetermined contact information provided by the patient to the testing facility. The health care practitioner computer is also configured to direct a health care practitioner critical data display to appear on the health care practitioner display monitor of a health care practitioner. The health care practitioner computer is further configured to prompt the health care practitioner through the patient critical data display to review the medical testing result for the patient appearing on the health care practitioner critical data display and designate the medical testing result as critical or noncritical for the patient. The health care practitioner computer is yet further configured to prompt the health care practitioner through the health care practitioner critical data display to enter patient instructions.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 schematically shows a critical data display system in an embodiment of the present disclosure.

FIG. 2 schematically shows a critical display screen in an embodiment of the present disclosure.

FIG. 3 schematically shows a critical display screen showing critical data in an embodiment of the present disclosure.

FIG. 4 schematically shows a top view and a perspective view of a critical display screen in an embodiment of the present disclosure.

FIG. 5 schematically shows a process flow path in an embodiment of the present disclosure.

FIG. 6 schematically shows a noncritical result process flow path in an embodiment of the present disclosure.

FIG. 7 schematically shows a critical result process flow path in an embodiment of the present disclosure.

FIG. 8 schematically shows a flow process of a flow diagram of a patient representative interacting with a health care practitioner in an embodiment of the present disclosure.

FIG. 9 schematically shows a flow process of a flow diagram of a patient representative interacting with a health care practitioner with the aid of a data entry specialist in an embodiment of the present disclosure.

FIG. 10 schematically shows a flow process of a flow diagram of a patient representative interacting with a health care practitioner with the aid of an ambassador physician in an embodiment of the present disclosure.

FIG. 11 schematically shows a flow process of a flow diagram of a patient representative interacting with a health care practitioner with the aid of a data entry specialist and an ambassador physician in an embodiment of the present disclosure.

Wherever possible, the same reference numbers will be used throughout the drawings to represent the same parts.

### DETAILED DESCRIPTION OF THE INVENTION

Provided are computer-implemented critical data display systems, display methods, and displays for facilitating communication between health care practitioners and their patient representatives, especially with regard to critical medical testing results.

Embodiments of the present disclosure, for example, in comparison to concepts failing to include one or more of the features disclosed herein, facilitate direct electronic communication between patient and health care practitioner, organize access to medical testing results on a single easy-to-use display screen, highlight critical medical testing results customized for the individual patient representative, improve the communication between patient representative and health care practitioner about the patient's critical results from medical testing, provides customized instructions from the health care practitioner to the patient representative based on the medical testing results, provide an inexpensive health care concierge service for medical testing results, or combinations thereof.

In exemplary embodiments, the critical data display system is a computer-implemented system.

A recently-conducted survey of nearly 1000 randomly-selected individuals assessed their satisfaction with their current critical information communication with their provider. 15% of respondents either themselves had a serious medical condition or cared for someone who did. A majority of those who had a current health care portal were willing to pay up to about ten times more to have a communication tool having features of the critical data display system described herein than those individuals who currently have no electronic medical record portal. This result was completely unexpected and even initially counterintuitive. The United States government has spent $10 billion to improve health care communication between patient and providers, and yet it is precisely those individuals who have electronic medical records, not those without electronic medical records and medical record portals, who are much more interested in using and paying for a health care portal with features of the critical data display system disclosed herein. These efforts have largely failed, because patients and their health care proxies (HCP) want more guidance not more information. There is currently no simple direct communication between a patient and his or her health care practitioner.

Often overlooked is the real need of a patient to be comforted and directed by his or her health care practitioner. The needs of the patient have been simultaneously recognized and thwarted by the government. HIPAA regulations give infinite power to patients over the control of their medical record. This patient's health care practitioner, however, is constrained in the paths to communicate with the patient without violating HIPAA rules. Both the medical entity that the health care practitioner works for and the electronic record system share the same concerns about HIPAA compliance.

When a health care proxy or other patient representative is attending to and helping the patient to receive proper health care, the patient representative may have the further added stress of not having access to the medical records of the patient. In some embodiments, the critical queries are initiated by the patient. The health care practitioner receives the result. The above-noted survey shows that patients with health care portals are more likely to want to use a display system having one or more of the features disclosed herein than those who do not have a portal. The use of an electronic portal paradoxically increases rather than decreases the patient representative's need for guidance from the health care practitioner.

In some embodiments, the health care proxy or other patient representative may communicate through the critical data display system instead of the patient. For example, the patient representative may discuss a worrisome lab result with the patient and upload the lab result to the critical data display system. The ambassador physician may receive the lab results through the critical data display system and send an immediate response to the patient representative, either directly or through the critical data display system. The patient representative may share that immediate response with the patient. The ambassador physician may then share the lab results and the immediate response with the health care practitioner through the critical data display system and can help onboard the health care practitioner. The health care practitioner may then participate in the communication with the patient representative through the critical data display system.

As used herein, the term “patient representative” may refer to the patient himself or herself, a legal guardian of the patient, a health care proxy of the patient, or any other patient designee whom the patient has legally authorized to receive the patient's medical records and discuss the patient's medical records with the patient's health care practitioner.

As used herein, the term “health care practitioner” may refer to a primary care physician of the patient, a specialist physician of the patient, or any other physician of the patient.

The health care practitioner's communication efforts are constrained by the different goals of the medical entity that the provider works for, the electronic health record (EHR) system that the health care practitioner uses and the need to preserve the confidentiality of the health record as required by HIPAA. The issues of data security and commercial interests become conflated. Fast health interoperability resources (FHIR) first emerged in 2011 but is still being modified and is mandated for universal use. There are two further problems. First, there is a need for a common, simple, and intuitive display that can health care be used across systems that are interoperable. U.S. Pat. No. 8,489,544 and/or U.S. Pat. No. 9,286,443 provide such a solution. There is a further problem, however, that, absent a patient waiver, the prompt and easy communication of a tiny, but vital and critical, fraction of the total data record between patient and health care practitioner cannot occur, much to the frustration of both patient and health care practitioner. By having patients waive their HIPAA rights and transmit the results in question via the critical data display system described herein to the health care practitioner for review, this data transfer can occur.

In some embodiments, the health care practitioner must accept the transfer. To achieve this critical data acceptance, a medical intermediary, or “ambassador physician” (AP), may review and provide guidance to the patient on the information in the critical data display system and reset the critical values for this patient to reflect the patient's current condition. The AP preferably provides only these important but limited services in the process and has no interest in replacing the health care practitioner. This information is then transmitted by the AP through the critical data display system to the patient's health care practitioner and the patient's chart with a link so that the health care practitioner, ideally, signs on to the critical data display system and assumes the entire role of the AP, or at least to set the critical values for the patient. The difference from a telehealth second opinion or even telehealth visit is that there is no future contact with the AP possible for the patient. In some such embodiments, the critical data display system described herein is more like a remote review of an x-ray test that will likely be revisited by a local physician the following morning or later.

In some embodiments, the AP is anonymous to the patient, in part, so that the health care practitioner need not threatened by the involvement of another practitioner in the care of the health care practitioner's patient. This structure is valuable and reassuring to the health care practitioner. A second opinion initiated by the patient without first consulting the health care practitioner can lead to health care practitioner stress. For example, the second opinion practitioner may recommend face-to-face consultation with another practitioner, possibly transfer of care or even question the adequacy of the care the health care practitioner has delivered. At the very least there is a higher level of doubt on the part of the health care practitioner for the patient's good will towards the health care practitioner. The health care practitioner may look then to initiate transfer of the patient's care.

In exemplary embodiments, a critical data display system permits the patient to designate a patient representative, who receives access equivalent to the patient's access to the correspondences and data in the critical data display system. Alternatively, the patient representative may provide paperwork to the critical data display system sufficient for the patient representative to receive equivalent access. In the case of a minor patient, the patient's legal guardian may automatically be designated as a patient representative to receive access equivalent to the patient's access in the critical data display system.

In exemplary embodiments, a critical data display system addresses the previously-mentioned factors that obstruct optimal direct communication between a patient representative and the patient's health care practitioner first by obtaining the patient's consent to share his/her critical medical information with the critical data display system and thus the transaction is HIPAA compliant. The patient representative identifies a concerning result or condition and forwards the result to the critical data display system. If there is electronic connection available to the patient representative to transmit the document to the critical data display system, the electronic connection is used. If there is no electronic connection to the provider from the patient representative, the report is sent via an email. The email may be digitized either by ocular cypher recognition (OCR) or alternatively manually. The report is then delivered to an AP who works as locum tenens for the critical data display system. The AP provides provisional advice to the patient representative about what to do. The AP then sends notification via fax to the patient's health care practitioner. The notification, and any subsequent notifications, includes a link that encourages the patient's health care practitioner to:
1. Optimally take over the critical data display system role himself/herself and both instruct and reset critical values as needed for that patient's specific needs (for example elevating white cell count in a patient with chronic lymphocytic leukemia);
2. Allow the AP to continue to communicate with the patient representative but allow the health care practitioner to reset any patient specific critical values; or
3. Allow the AP to set the critical values and communicate with the patient representative.

A goal of the critical data display system throughout is to strengthen the relationship between the patient and the health care practitioner. By having an ambassador physician who is not and never will be a health care practitioner to the patient and will not arrange for a second opinion for the patient, the health care practitioner is not threatened by the critical data display system. Providing a second opinion service that the health care practitioner does not suggest would likely been seen as very threatening, but the role of the AP is like that of a provider at a patient visit to an urgent care center to help the health care practitioner care for the patient.

In some embodiments, a critical data display system connects the patient representative participation in the critical data display system with the patient's health care practitioner by the patient HIPAA-enabling the health care practitioner to receive a complete set of the patient's medical records in real time, with the critical parameters being designed by the health care practitioner to fit the patient's specific set of medical problems. In such embodiments, a computer-knowledgeable front office staff person for the health care practitioner may serve as a facilitator of the critical data display system connection/relationship between the patient representative, who may not be computer savvy and may be very distracted or stressed, and the health care practitioner, who may also be not computer savvy and will likely be very busy with a myriad of other concerns. This facilitator may serve as a contact point for urgent patient representative questions in addition to critical results via the critical data display system.

Computer-implemented systems, methods, and displays improve the electronic communication between patient representative and health care practitioner about the patient's critical results from medical testing. The systems, methods, and displays and display are patient-driven. The patient owns the medical data and, through the systems, methods, and displays, select whether to share it with a health care practitioner, thereby satisfying HIPAA regulations.

In some embodiments, the systems, methods, and displays gather and permit access to records across multiple electronic medical record (EMR) systems. In some embodiments, the systems, methods, and displays facilitate the interaction by putting the health care practitioner in a concierge position, such that the health care practitioner can set critical medical testing result values through the critical data display process and display that are specific for the patient based on the patient's history. The patient representative benefits through real-time communications with the health care practitioner through a single-screen display interface and knows how to act when critical results come up, such as, for example, going to an emergency room for treatment.

In some embodiments, the critical data display system is EMR-agnostic and receives results directly from the medical testing facility. In some embodiments, the critical data display system receives direct communication between the patient representative and a single health care practitioner and puts the patient representative in control of the patient's medical testing data, which is shared with the health care practitioner. In some embodiments, the critical data display system provides a superior display interface that is easy to understand and that stacks multiple records in a single display screen, which facilitates triage of data.

In exemplary embodiments, a patient accesses the critical data display system through a unique login and password. Within the critical data display system, the patient selects a single predetermined health care practitioner and authorizes sharing of medical data with the health care practitioner.

In some embodiments, the patient has, authorizes, or designates one or more patient representatives, who are granted similar access and capabilities to the critical data display system through their own unique login and password. This patient representative may then send and receive communications with the health care practitioner and have access to the patient critical data display through the critical data display system in a similar manner to the patient. In such situations, the patient may be a minor, may be unable to make their own medical decisions, or may merely have authorized another person as his or her patient representative to communicate with the health care practitioner and aid the patient in getting the medical care that he or she needs.

In some embodiments, when a patient has medical testing done, the critical data display system allows the patient to decide whether or not to have the results of that medical testing sent to a single predetermined health care practitioner, such as, for example, a doctor, a nurse, or a medical specialist, based on whether or not the patient provides predetermined contact information to the testing lab or facility. In exemplary embodiments, the predetermined contact information is unique for the patient. In some embodiments, the predetermined contact information is a facsimile number. In some embodiments, the predetermined contact information is an email address.

If the patient chooses to provide the predetermined contact information, the critical data display system may directly receive the patient's electronic medical testing results from the lab conducting the medical testing based on that contact information, thereby satisfying HIPAA regulations. In some embodiments, the critical data display system receives, digitizes, and categorizes the electronic medical testing results as either within normal ranges or outside of normal ranges, such as, for example, critical or critically abnormal, based on either a standard or customized criterion for criticality and then transmits the medical testing results electronically to the single predetermined health care practitioner designated by the patient in the critical data display system and displays the results on a screen of the health care practitioner with any critically abnormal result being highlighted.

In some embodiments, the health care practitioner reviews the displayed results, optionally adjusts the critical criterion for any medical testing results to customize for the patient, approves the displayed results, and provides instructions for the patient to the system, based on the medical testing results. Thus, the critical data display system gives the health care practitioner the authority, independence, and flexibility to take care of a patient's specific needs, a level of autonomy not currently offered in electronic healthcare system.

In some embodiments, the critical data display system then displays the critically abnormal results on a screen to the patient, as approved by the single health care practitioner, on a single display, along with guidance or patient instructions from the health care practitioner on what next step or what further actions the patient should take. In some embodiments, the critical data display system only displays critical medical testing results to the patient and not non-critical results. In other embodiments, the patient may access from the display all medical testing results for the patient in the critical data display system, but the display highlights or otherwise directs the patient's attention only to the critical medical testing results.

Appropriate patient instructions to be provided by the health care practitioner through the critical data display system may include, but are not limited to, scheduling a follow-up appointment after a predetermined period of time, scheduling an appointment for further medical testing, taking medication, or seeking immediate medical attention, such as, for example, calling 911 or going to an emergency room.

The patient may benefit, for example, from knowing which results matter from a set of medical testing results, being able to access the results on any type of electronic device, receiving an increased quality of care, and/or receiving direct instructions from the health care practitioner.

The health care practitioner may benefit, for example, from being able to set critical parameters for each category of medical testing results based on the specific needs of the patient, being able to see all critical results on a single screen regardless of the number of patients, being able to export results to a portable document format (PDF), being more easily able to meet the direct needs of individual patients, and/or being able to triage critical results of patients.

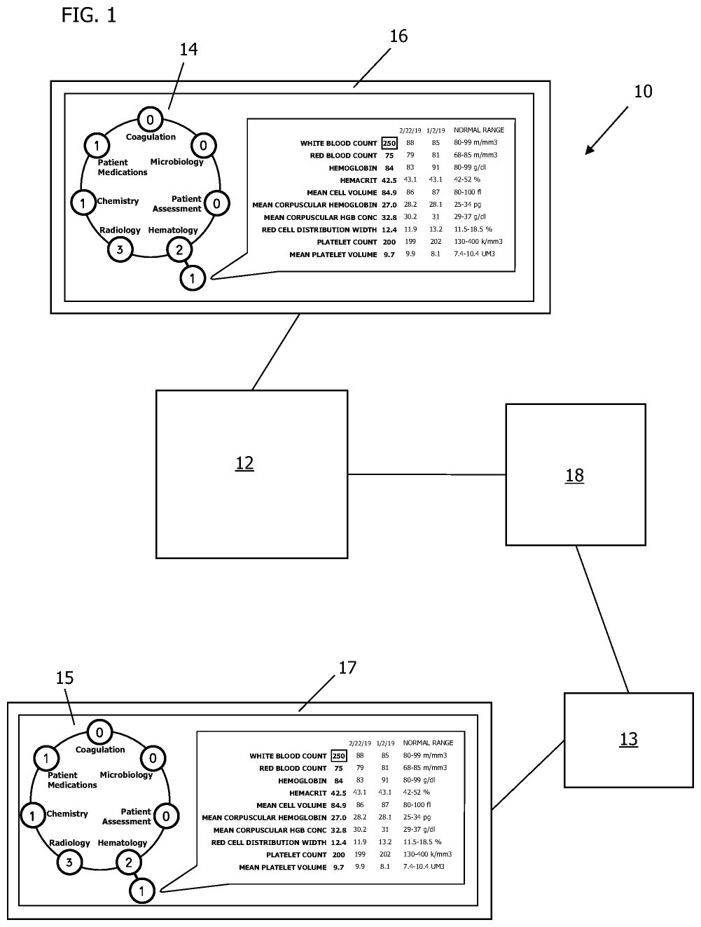

Referring to FIG. 1, a critical data display system 10 includes a patient local computer 12 enabling a patient critical data display 14 to a patient display monitor 16, a practitioner local computer 13 enabling a practitioner critical data display 15 to a practitioner display monitor 17, and one or more server computers 18 in electronic communication with the patient local computer 12 and the practitioner local computer 13. The patient local computer 12 receives inputs from a patient representative. The practitioner local computer 13 receives inputs from a health care practitioner. The local computers 12, 13 may be on networks and in electronic or wireless communication with the server computers 18, where a database of the medical testing results may be maintained, or with other local computers (not shown). In exemplary embodiments, the critical data display system 10 includes a critical data display program that implements one or more critical data display processes.

A local computer 12, 13, as used herein, may refer to a laptop computer, a desktop computer, an electronic tablet, a smart phone, or any other electronic device capable of processing data, receiving, and processing designated user inputs, and directing the display of an image on an electronic display monitor 16, 17. In some embodiments, the critical data display system 10 includes an app that runs on the local computers 12, 13. In some embodiments, the critical data display system 10 is web-based.

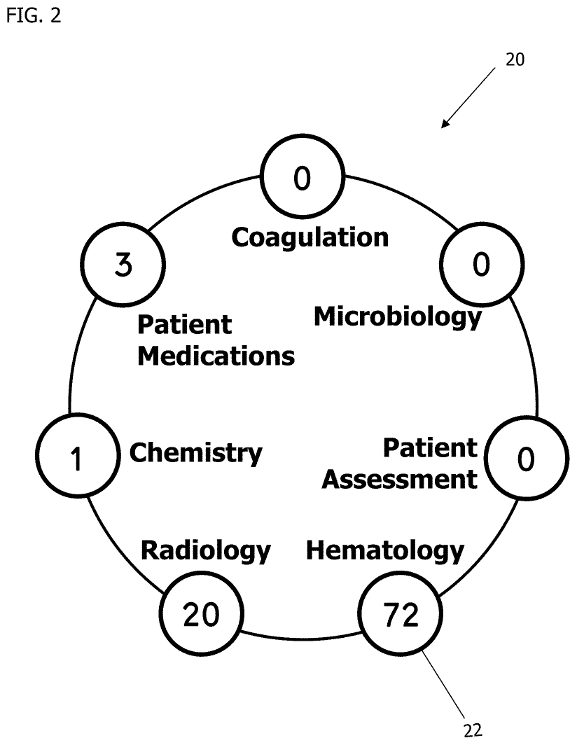

FIG. 2 shows patient data on a practitioner display circle 20 for a health care practitioner. In some embodiments, the practitioner display circle 20 represents all data for a single patient of the health care practitioner provided through the critical data display system 10. In other embodiments, the practitioner display circle 20 represents data for multiple patients, up to all of the patients who have selected the health care practitioner as their single health care practitioner in the critical data display system 10. The practitioner display circle 20 preferably persists on the practitioner display monitor 17 as a point of reference no matter what the practitioner is reviewing in the other portion of the practitioner display monitor 17. The practitioner display circle 20 allows the practitioner to prioritize his or her review of the data. Each type of data is grouped with similar data types. The numeral in each type circle 22 on the practitioner display circle 20 represents the number of different reports of each type. The numeral in each smaller circle (not shown in FIG. 2) extending radially off the practitioner display circle 20 on the perimeter of the practitioner display circle 20 represent a subset of the reports of that type. In some embodiments, the type circle 22 includes all data of that type. In other embodiments, the type circle 22 includes only all non-critical data of that type. In yet other embodiments, the type circle 22 includes only all unreviewed data of that type. In some embodiments, the smaller circle includes only all unreviewed data of that type. In other embodiments, the smaller circle includes only all critical data.

Although the practitioner display circle 20 includes seven type circles 22, any number of type circles 22 may be used to correspond to an appropriate number of medical testing types. The type circles 22 preferably are always arranged in the same order so that the practitioner always knows where to look for a certain type of data.

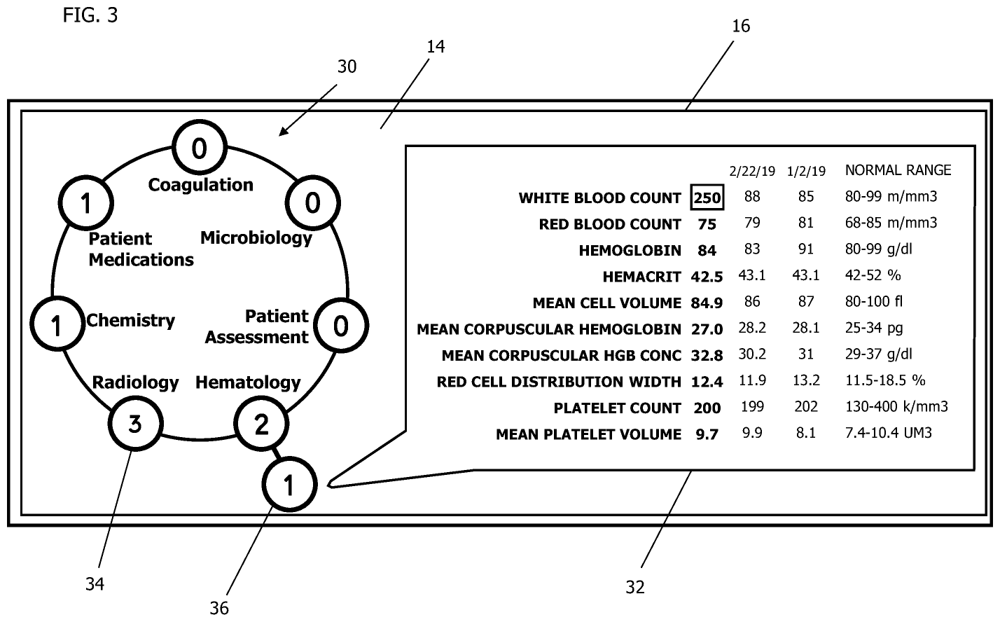

FIG. 3 shows a patient critical data display 14 on a patient display monitor 16 displaying patient data for the single patient to the patient representative. The left portion of the display area presents a patient display circle 30. The right portion of the display area presents the details of patient data 32 for a specific medical testing result or other medical data of the patient. The details of patient data 32 shows both a normal range for the medical testing data and the patient's most recently measured value and may also include historical values, if any. The critical value is highlighted in the details of patient data 32.

The patient display circle 30 preferably persists on the display screen as a point of reference no matter what the patient representative is reviewing in the other portion of the screen. The patient display circle 30 allows the patient representative to prioritize his or her review of the data. Each type of data is grouped with similar data types. The numeral in each type circle 34 represents the total number of different reports of each type. The numeral in each smaller circle 36 extending radially off the display circle 30 on the perimeter of the patient display circle 30 represents a subset of the reports of that type that include critical data.

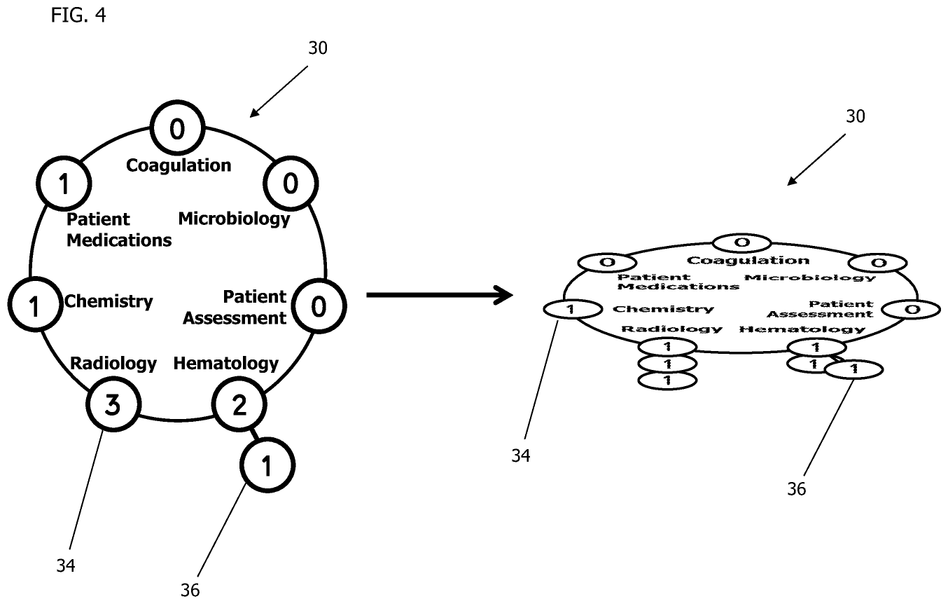

FIG. 4 shows a top view of the patient display circle 30 of FIG. 3 on the left side and a perspective view of the patient display circle 30 on the right side showing the stacking represented by the numerals in the type circles 34 and smaller circles 36. All and only critical results are displayed outside the primary circle. The display circle 30, type circles 34, and smaller circle 36 appear as ellipses on the right side of FIG. 4 only to reflect the schematic perspective from which they are being viewed.

The layout of the practitioner display circle 20 may be the same as or different from the layout of the patient display circle 30.

In addition to the features and functionalities described herein, the health care practitioner display system of the critical data display system 10 may incorporate one or more features or functionalities of the triaged data display system of U.S. Patent Application Publication No. 2020/0005917, which is incorporated by reference in its entirety.

In addition to the features and functionalities described herein, both the patient display system and the health care display system of the critical data display system may incorporate one or more features or functionalities of the systems of U.S. Pat. No. 8,489,544 or U.S. Pat. No. 9,286,443, which are both incorporated by reference in their entireties.

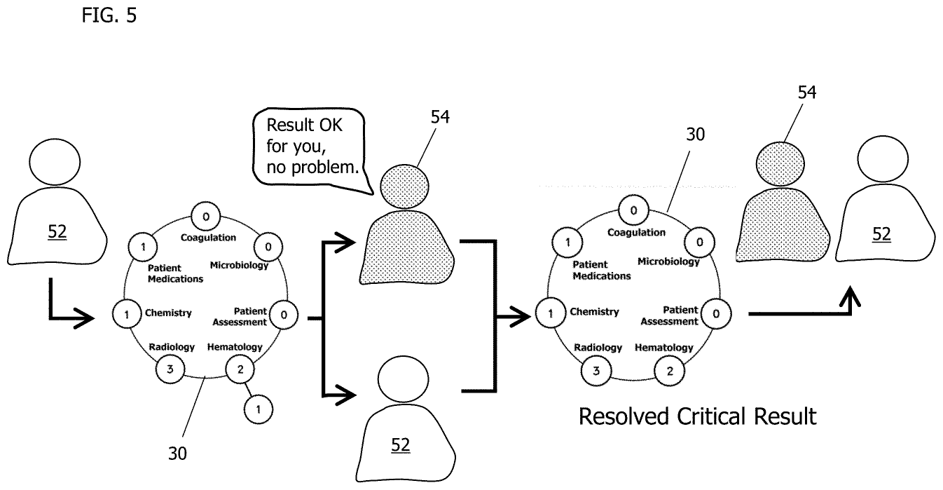

FIG. 5 shows a flow diagram 50 schematically representing a process of resolving a critical medical testing result. The patient representative 52 authorizes the testing facility to send the medical testing results to the single health care practitioner 54 by providing the predetermined contact information to the testing facility. The testing facility sends the medical testing results using the provided predetermined contact information. The critical data display system 10 electronically receives the medical testing results and digitizes and evaluates the resulting data with respect to general critical criteria. The critical data display system 10 may alternatively send the data to an ambassador physician (not shown in FIG. 5) who evaluates the resulting data with respect to general critical criteria and may recommend adjusting the critical range based on the patient's specific condition or medical history. The critical data display system then transmits those results to the predetermined single health care practitioner 54 on the critical data display 15 of the practitioner display monitor 17 with any critical results being highlighted.

The single health care practitioner 54 reviews the data on the practitioner display monitor 17, customizes any critical criteria to the patient, determines whether any of the data represents a critical condition for the individual patient, and provides instructions for the patient. The critical data display system then transmits the practitioner-modified results and instructions to display on the patient display monitor 16 with any critical results being highlighted.

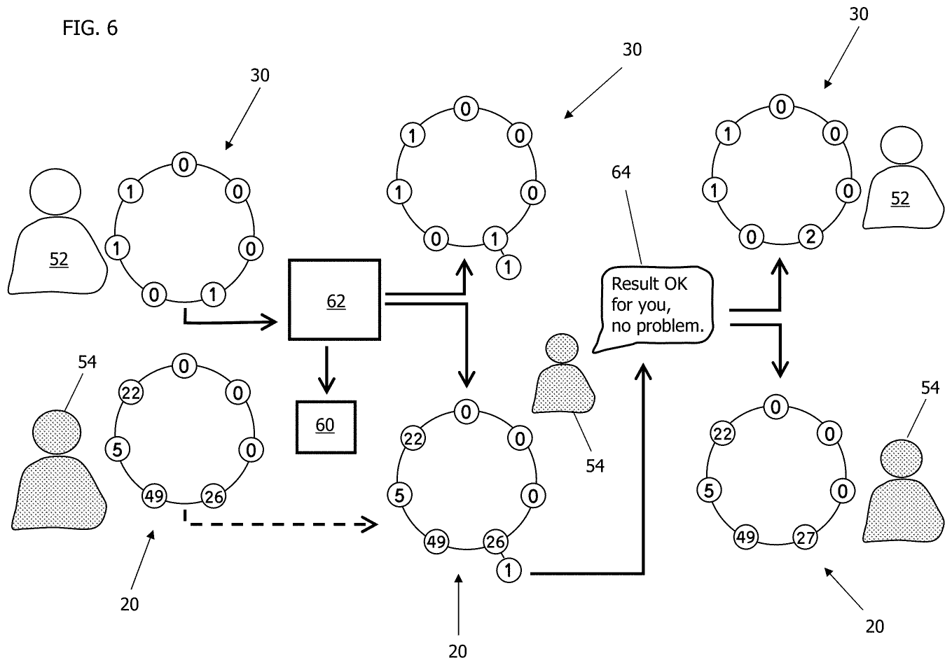

FIG. 6 shows in more detail steps in processing new medical testing results 60, such as, for example, in the form of an electronic medical record (EMR), from lab testing 62 that are initially categorized as including one or more critical values, but when evaluated by the health care practitioner 54, who customizes for the particular patient, the results no longer include critical values. The health care practitioner 54 communicates patient instructions 64 to the patient representative 52 through the critical data display system that no further action is needed by the patient at this time. In some embodiments, the patient instructions 64 appear on the patient critical data display 14.

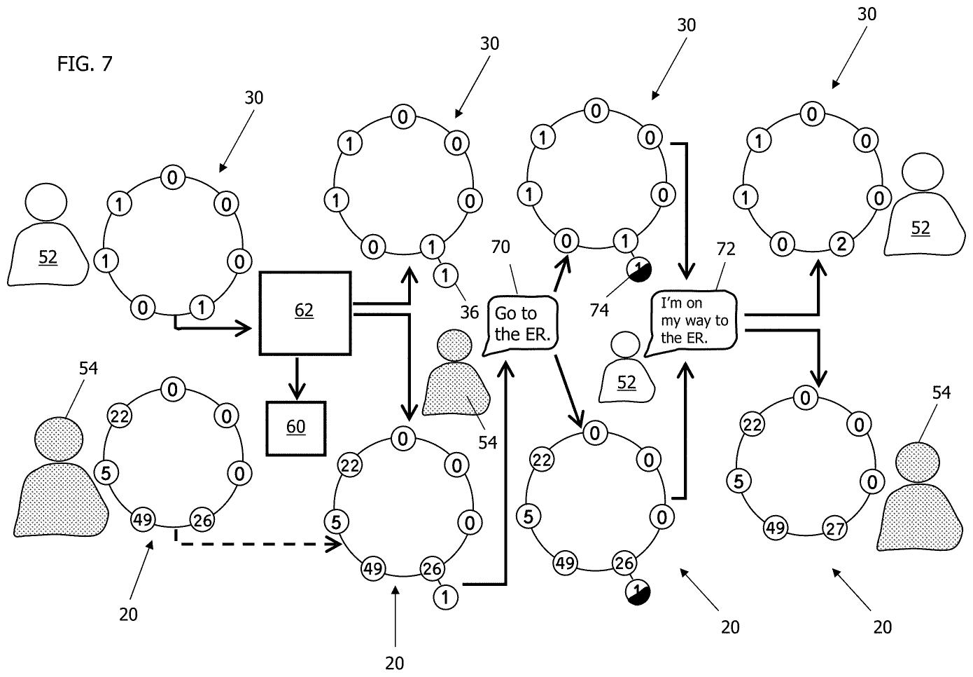

FIG. 7 shows in more detail the steps in processing new medical testing results 60 from lab testing 62 that are initially categorized as including one or more critical values, and when evaluated by the health care practitioner 54, who customizes for the particular patient, the results still include one or more critical values. The health care practitioner 54 communicates patient instructions 70 to the patient representative 52 through the critical data display system that action needs to be taken by the patient, in this case an immediate trip to the emergency room. In some embodiments, the patient instructions 70 appear on the patient critical data display 14. The patient representative 52 may enter a patient reply 72, which may then appear on the practitioner critical data display 15, confirming that the patient representative 52 saw the instructions and acted accordingly.

In some embodiments, as shown in FIG. 7, critical results on the patient display circle 30 appear differently before and after the health care practitioner has reviewed the medical testing results. For example, the small circle 36 is open on the patient display circle 30 prior to review by the health care practitioner but the small circle 74 becomes half-filled if still critical after review by the health care practitioner. In other embodiments, the patient display circle 30 initially shows all new medical testing results as non-critical prior to review by the health care practitioner so as to not unnecessarily alarm the patient representative 52 over critical results that are false positives.

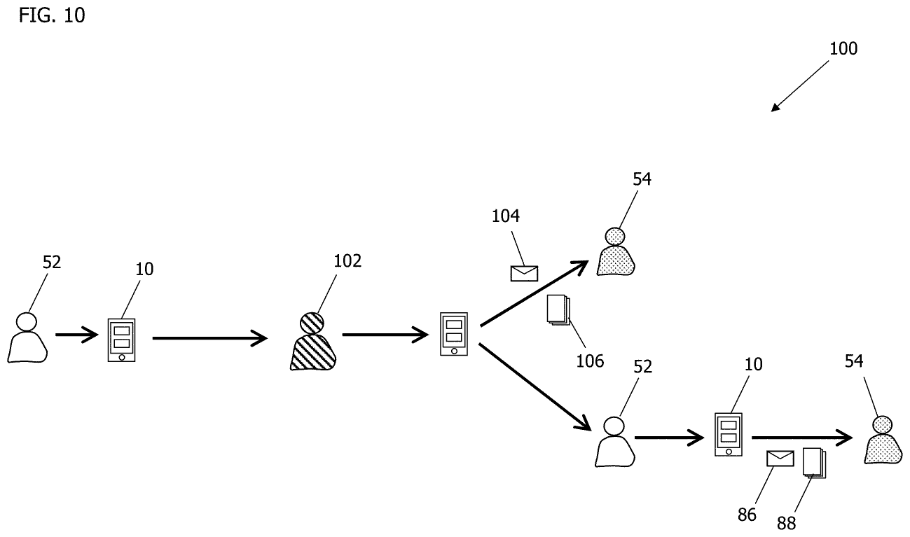

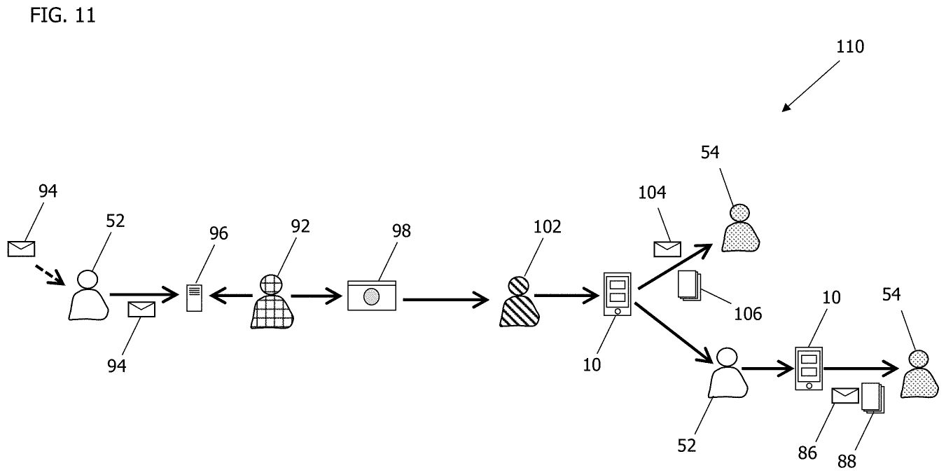

In other embodiments, an ambassador physician rather than the health care practitioner 54 of FIG. 5, FIG. 6, or FIG. 7, may preliminarily approve or reject the medical testing results 60, adjust the critical values, and/or provide patient instructions 70 through the critical data display system 10. The ambassador physician's assessment may then be provided to the patient representative 52 through the critical data display system 10, as shown in FIG. 10 and FIG. 11, or may alternatively first go to the health care practitioner 54, who may agree or disagree with the ambassador physician's assessment. In such cases, the critical data display system 10 may send only the assessment from the health care practitioner 54 to the patient representative 52.

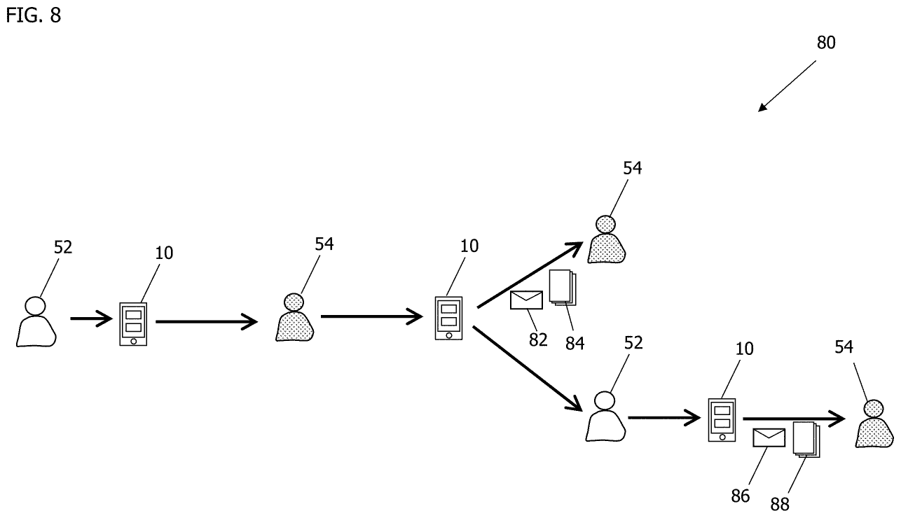

FIG. 8 shows a flow diagram 80 for the patient representative 52 interacting with the health care practitioner 54 without the aid of an ambassador physician or a data entry specialist. The patient representative 52 uploads a medical report, such as an xml report, to the critical data display system 10, which automatically parses the report and adds the results to the provider critical data display of the health care practitioner 54. The health care practitioner 54 reviews the test results through the provider critical data display, which may include accepting or rejecting the report, setting or adjusting critical ranges, and inputting feedback on the report. The feedback is sent through the critical data display system 10 to the patient critical data display and the report status is updated to indicate feedback has been provided. The critical data display system 10 sends an email notification 82 and a facsimile 84 to the health care practitioner 54 that includes the details of the interaction. An invitation link to the health care practitioner 54 is not needed, because the health care practitioner 54 is already a registered user of the critical data display system 10. The patient representative 52 views the feedback from the health care practitioner 54 and can acknowledge receipt to the critical data display system 10. The critical data display system 10 then sends an email notification 86 and a facsimile 88 to the health care practitioner 54 of the acknowledgment from the patient representative 52.

In some embodiments, the critical data display system 10 permits certain access and abilities to a data entry specialist associated with the health care practitioner. This data entry specialist may be located at the front desk of the health care practitioner's office. In conventional systems, there are often problems for both the patient representative and the health care practitioner in enabling software that does not even directly link the health care practitioner and patient representative, to the great frustration of the data entry specialist, who, in conventional systems, cannot facilitate reshaping, even with the health care practitioner's consent, this patient representative/practitioner interaction.

The access and abilities granted by the critical data display system 10 may include enabling the data entry specialist to sign off to adjust the communication between the health care practitioner and the patient representative. For example, the health care practitioner may provide patient-customized critical ranges or a message for the patient representative, either orally or in writing, to the data entry specialist, who is granted the ability to enter such information on behalf of the health care practitioner into the critical data display system 10. This is a very attractive feature for the data entry specialist, and the health care practitioner, the patient representative, and the critical data display system 10 benefit as well. The data entry specialist thus may serve as an intermediary to troubleshoot the interaction between the patient representative and the health care practitioner through the critical data display system 10.

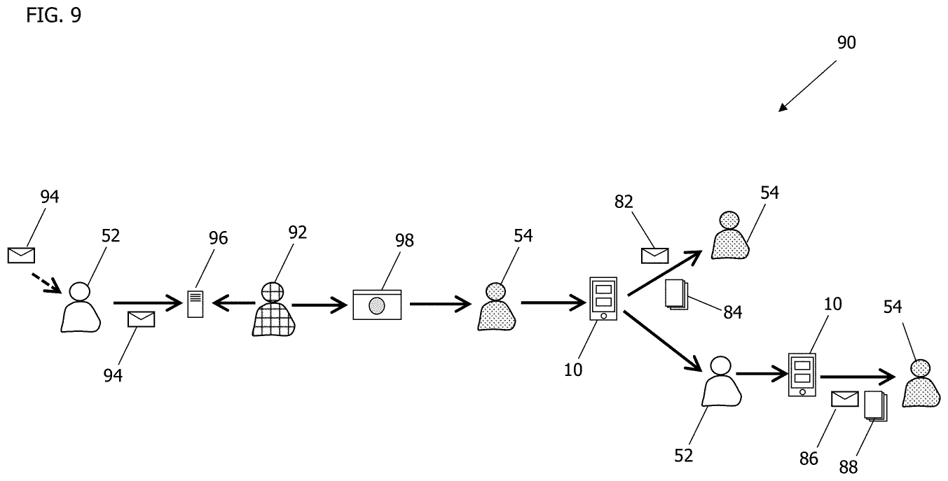

FIG. 9 shows a flow diagram 90, where a data entry specialist 92 aids in the interaction between the patient representative 52 and the health care practitioner 54 through the critical data display system 10. The patient representative 52 may receive a test result by email 92 and forward the email 94 to the critical data display system 10, which causes the email 94 to display in the inbox 96 of the data entry specialist 92. The data entry specialist 92 then enters the report 98 for the patient into the critical data display system 10, such as through a data entry application. When the report 98 is created in this manner, its status is “processing” at this point. Once the data is entered, the data entry specialist 92 changes the status of the report 98 to “ready for health care practitioner”. A link between the patient representative 52 and the specific health care practitioner 54 already exists in the critical data display system 10, so the report 98 is automatically assigned to the health care practitioner 54 by the critical data display system 10. The health care practitioner 54 reviews the report 98 through the practitioner critical data display, which may include accepting or rejecting the report, setting or adjusting critical ranges, and inputting feedback on the report. The feedback is sent through the critical data display system 10 to the patient critical data display and the report status is updated to indicate feedback has been provided. The critical data display system 10 sends an email notification 82 and a facsimile 84 to the health care practitioner 54 that includes the details of the interaction. The patient representative 52 views the feedback from the health care practitioner 54 and can acknowledge receipt to the critical data display system 10. The critical data display system 10 then sends an email notification 86 and a facsimile 88 to the health care practitioner 54 of the acknowledgment from the patient representative 52.

FIG. 10 shows a flow diagram 100, where an ambassador physician 102 aids in the interaction between the patient representative 52 and the health care practitioner 54 through the critical data display system 10. The patient representative 52 uploads a medical report, such as an xml report, to the critical data display system 10, which automatically parses the report and adds the results to the ambassador physician 102. The ambassador physician 102 reviews the test results. This review may include accepting or rejecting the report, setting or adjusting critical ranges, and inputting feedback on the report. The feedback is sent through the critical data display system 10 to the patient critical data display, and an email notification 104 and a facsimile 106 are sent to the health care practitioner 54 that includes the details of the interaction and a custom invitation link to view the report. The report status is updated to indicate feedback has been provided. The patient representative 52 views the feedback from the ambassador physician 102 and can acknowledge receipt to the critical data display system 10. The critical data display system 10 then sends an email notification 86 and a facsimile 88 to the health care practitioner 54 of the acknowledgment from the patient representative 52.

FIG. 11 shows a flow diagram 110, where a data entry specialist 92 and an ambassador physician 102 aid in the interaction between the patient representative 52 and the health care practitioner 54 through the critical data display system 10. The patient representative 52 may receive a test result by email 92 and forward the email 94 to the critical data display system 10, which causes the email 94 to display in the inbox 96 of the data entry specialist 92. The data entry specialist 92 then enters the report 98 for the patient into the critical data display system 10, such as through a data entry application. When the report 98 is created in this manner, its status is “processing” at this point. Once the data is entered, the data entry specialist 92 changes the status of the report 98 to “ready for health care practitioner”. The critical data display system 10 then provides the report to the ambassador physician 102, who reviews the test results. This review may include accepting or rejecting the report, setting or adjusting critical ranges, and inputting feedback on the report. The feedback is sent through the critical data display system 10 to the patient critical data display, and an email notification 104 and a facsimile 106 are sent to the health care practitioner 54 that includes the details of the interaction and a custom invitation link to view the report. The report status is updated to indicate feedback has been provided. The patient representative 52 views the feedback from the ambassador physician 102 and can acknowledge receipt to the critical data display system 10. The critical data display system 10 then sends an email notification 86 and a facsimile 88 to the health care practitioner 54 of the acknowledgment from the patient representative 52.

In exemplary embodiments, the local computers 12, 13, the server computer 18, and any other electronic devices in electronic communication as part of the critical data display system 10 include respective sets of internal components and external components. Each of the sets of internal components may include one or more operating systems, one or more computer-readable tangible storage devices, one or more processors, one or more computer-readable random access memories (RAMs), and one or more computer-readable read-only memories (ROMs) on one or more buses. The operating system and a critical data display program are stored on one or more of the computer-readable tangible storage devices for execution by one or more of the processors via one or more of the RAMs. Each of the computer-readable tangible storage devices may be a magnetic disk storage device of an internal hard drive or a semiconductor storage device such as ROM, erasable programmable ROM (EPROM), flash memory, or any other computer-readable tangible storage device that can store a computer program and digital information.

Each set of internal components also includes a read/write (R/W) drive or interface to read from and write to one or more portable computer-readable tangible storage devices, such as a compact disc read-only memory (CD-ROM), digital versatile disc (DVD), memory stick, magnetic tape, magnetic disk, optical disk, or semiconductor storage device. The critical data display program may be stored on one or more of the portable computer-readable tangible storage devices, read via R/W drive, or interface and loaded into a hard drive.

Each set of internal components may also include a network adapter or interface such as a transmission control protocol/internet protocol (TCP/IP) adapter card. The critical data display program may be downloaded to the local computers 12, 13 and/or server computer 18 from an external computer via a network (for example, the Internet, a local area network (LAN) or other, wide area network (WAN)) and network adapter or interface. From the network adapter or interface, the critical data display program may be loaded into the hard drive. The network may include copper wires, optical fibers, wireless transmission, routers, firewalls, switches, gateway computers, and/or edge servers.

Each of the sets of external components may include a display monitor 16, 17, a keyboard, and/or a computer mouse. Each of the sets of internal components may also include device drivers to interface to the display monitor 16, 17, the keyboard, and the computer mouse. The device drivers, R/W drive, or interface and network adapter or interface include hardware and software stored in a storage device and/or ROM.

The critical data display program may be written in any of various programming languages including low-level, high-level, object-oriented, or non-object-oriented languages. Alternatively, the functions of the critical data display program may be implemented in whole or in part by computer circuits and other hardware (not shown).

A critical data display program product may include a computer-readable storage medium (or media) having computer-readable program instructions thereon for causing a processor to carry out aspects of the critical data display processes described herein.

The computer-readable storage medium may be a tangible device that retains and stores instructions for use by an instruction execution device. The computer-readable storage medium may be, for example, an electronic storage device, a magnetic storage device, an optical storage device, an electromagnetic storage device, a semiconductor storage device, or any suitable combination of the foregoing. More specific examples of appropriate computer-readable storage media include a portable computer diskette, a hard disk, a RAM, a ROM, an EPROM, a static random access memory (SRAM), a portable CD-ROM, a DVD, a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media, or electrical signals transmitted through a wire.

Computer-readable program instructions may be downloaded to respective computing/processing devices from a computer-readable storage medium or to an external computer or external storage device via a network, for example, the Internet, a LAN, a WAN, and/or a wireless network. The network may include copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the critical data display system may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language, such as Smalltalk or C++, and procedural programming languages, such as the “C” programming language or similar programming languages. The computer-readable program instructions may execute entirely on the local computers 12, 13, partly on the local computers 12, 13, as a stand-alone software package, partly on the local computers 12, 13 and partly on a server computer 18, or entirely on the server computer 18. In the latter scenario, the server computer 18 may be connected to the local computers 12, 13 through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA), may execute the computer-readable program instructions by utilizing state information of the computer-readable program instructions to personalize the electronic circuitry, in order to perform aspects of the critical data display processes.

It will be understood that each aspect of the critical data display processes may be implemented by computer-readable program instructions. These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing steps of the critical data display processes. These computer-readable program instructions may also be stored in a computer-readable storage medium that may direct a computer, a programmable data processing apparatus, and/or other devices to function in a particular manner, such that the computer-readable storage medium having instructions stored therein includes an article of manufacture including instructions that implement aspects of the critical data display processes.

The computer-readable program instructions may also be loaded onto a computer, other programmable data processing apparatus, or other device to cause a series of operational steps to be performed on the computer, other programmable apparatus, or other device to produce a computer-implemented process, such that the instructions that execute on the computer, other programmable apparatus, or other device implement the specified functions/acts of the critical data display processes.

Although the critical data display process and critical data display system have been described with respect to medical records and for use in the medical field, the principles of the process and system may be implemented in other ways as well. In some embodiments, a critical data display system is implemented in a more modular way, such that the user interface (UI) may be used by different companies, such as, for example, a data visualization plugin for data analysis tools or spreadsheets, such as, for example, Microsoft Excel and Tableau. In some embodiments, certain functions are implemented separately, using a modular critical data display UI.

All above-mentioned references are hereby incorporated by reference herein.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A critical data display process comprising:
a computer directing a medical testing result for a patient to appear on a health care practitioner critical data display of a health care practitioner display monitor of a single health care practitioner;
the computer prompting the single health care practitioner through the health care practitioner critical data display to review the medical testing result for the patient on the health care practitioner critical data display, to designate or accept a previous designation of the medical testing result as either critical or noncritical, and to enter or authorize patient instructions; and
the computer directing a patient critical data display to appear on a patient display monitor of a patient representative of the patient, wherein the patient critical data display displays the medical testing result as either critical or noncritical as designated by the single health care practitioner and displays the patient instructions from the single health care practitioner.

2. The critical data display process of claim 1 further comprising the computer electronically receiving the medical testing result from a testing facility based on predetermined contact information provided by the patient to the testing facility.

3. The critical data display process of claim 1 further comprising the computer prompting the patient representative to approve transmitting medical records of the patient to the single health care practitioner.

4. The critical data display process of claim 1 further comprising, prior to the computer directing the medical testing result to appear on the health care practitioner critical data display of the health care practitioner display monitor of the single health care practitioner, the computer directing an ambassador physician to review the medical testing result for the patient on an ambassador physician critical data display and prompting the ambassador physician to approve or adjust a critical range for a parameter of the medical testing result based on a health history of the patient.

5. The critical data display process of claim 4, wherein the computer directing the medical testing result for the patient to appear on the health care practitioner critical data display of the health care practitioner display monitor of the single health care practitioner further comprises the computer displaying the medical testing result on the health care practitioner critical data display as preliminarily either critical or noncritical based on the critical range provided by the ambassador physician.

6. The critical data display process of claim 5 further comprising the computer prompting the single health care practitioner through the health care practitioner critical data display to accept or adjust the critical range for the parameter of the medical testing and to authorize the ambassador physician to.

7. The critical data display process of claim 1 further comprising, prior to the computer directing the medical testing result to appear on the health care practitioner critical data display of the health care practitioner display monitor of the single health care practitioner, the computer directing an ambassador physician to review the medical testing result for the patient on an ambassador physician critical data display and prompting the ambassador physician to preliminarily designate the medical testing result as either critical or noncritical.

8. The critical data display process of claim 7, wherein the computer directing the medical testing result for the patient to appear on the health care practitioner critical data display of the health care practitioner display monitor of the single health care practitioner further comprises the computer displaying the medical testing result on the health care practitioner critical data display as critical or noncritical as preliminarily designated by the ambassador physician.

9. A critical data display process comprising:
a computer prompting a patient representative of a patient through a patient display to authorize transmittal of medical records to a single health care practitioner;
the computer electronically receiving a medical testing result for the patient; and
the computer electronically transmitting the medical testing result to the single health care practitioner.

10. The critical data display process of claim 9, wherein the computer electronically receives the medical testing result for the patient from the patient representative.

11. The critical data display process of claim 9, wherein the computer electronically receives the medical testing result for the patient from a testing facility.

12. The critical data display process of claim 9 further comprising the computer electronically transmitting the medical testing result to an ambassador physician prior to transmitting the medical testing result to the single health care practitioner.

13. The critical data display process of claim 12 further comprising the computer prompting the ambassador physician through an ambassador physician critical data display to apply predetermined critical values and to suggest adjustments to the predetermined critical values based on a medical history of the patient.

14. The critical data display process of claim 13 further comprising the computer electronically transmitting the adjustments to the predetermined critical values suggested by the ambassador physician along with the medical testing result to the single health care practitioner.

15. A critical data display system comprising:
a server computer, the server computer being configured to:
electronically correspond with a heath care practitioner computer to receive a medical testing result reviewed and designated as critical or noncritical by a single health care practitioner and to receive a patient instruction provided by the single health care practitioner;
direct a patient critical data display to appear on a patient display monitor of a patient representative computer;
display the medical testing result on the patient critical data display as critical or noncritical as designated by the single health care practitioner; and
display the patient instruction on the patient critical data display.

16. The critical data display system of claim 15, wherein the server computer is further configured to request and receive authorization from the patient representative to transmit medical records of the patient to a single health care practitioner.

17. The critical data display system of claim 15, wherein the server computer is further configured to electronically transmit the medical testing result to an ambassador physician prior to transmitting the medical testing result to the single health care practitioner.

18. The critical data display system of claim 17, wherein the server computer is further configured to prompt the ambassador physician through an ambassador physician critical data display to apply predetermined critical values and to suggest adjustments to the predetermined critical values based on a medical history of the patient.

19. The critical data display system of claim 18, wherein the server computer is further configured to prompt the ambassador physician through an ambassador physician critical data display to apply predetermined critical values and to suggest adjustments to the predetermined critical values based on a medical history of the patient.

20. The critical data display system of claim 19, wherein the server computer is further configured to transmit the adjustments to the predetermined critical values suggested by the ambassador physician along with the medical testing result to the single health care practitioner.
